(19) United States
(12) Patent Application Publication
(10) Pub. No.: US 2011/0059487 A1
(43) Pub. Date: Mar. 10, 2011

(54) ENGINEERED MICROORGANISMS AND METHODS OF USE

(75) Inventors: Kevin A. Jarrell, Lincoln, MA (US); Gabriel Reznik, Waltham, MA (US); Michelle A. Pynn, Sharon, MA (US)

(73) Assignee: Modular Genetics, Inc., Cambridge, MA (US)

(21) Appl. No.: 12/822,765

(22) Filed: Jun. 24, 2010

Related U.S. Application Data
(60) Provisional application No. 61/220,186, filed on Jun. 24, 2009.
(60) Provisional application No. 61/291,434, filed on Dec. 31, 2009.

(51) Int. Cl. C12P 21/00; C12N 1/00; C12N 1/21; C12P 1/00; C12P 13/04

(52) U.S. Cl. 435/69.1; 435/243; 435/252.3; 435/252.31; 435/252.35; 435/41; 435/106

(57) ABSTRACT

The present invention provides, among other things, engineered microorganisms and methods that allow efficient conversion of soy carbohydrates to industrial chemicals by fermentation. In some embodiments, the invention provides microbial cells engineered to have increased efficiency in utilizing a soy carbon source (e.g., soy molasses, soy meal, and/or soy hulls) as compared to a parent cell. In some embodiments, microbial cells are engineered to have altered (e.g., increased) expression or activity of one or more carbohydrate modifying enzymes (e.g., glycosidases). In some embodiments, microbial cells are engineered to have altered localization of carbohydrate modifying enzymes (e.g., glycosidases). In some embodiments, engineered microbial cells provided herein are used to produce industrial chemicals (e.g., surfactin) using soy components as primary or sole carbon sources.

### CROSS REFERENCE TO RELATED APPLICATIONS

The present application claims priority to U.S. provisional patent application Ser. No. 61/220,186, filed Jun. 24, 2009, and to U.S. Provisional Application No. 61/291,434, filed Dec. 31, 2009, the entire contents of both of which are herein incorporated by reference.

### BACKGROUND

Soybeans are composed of about 37% protein, 18% oil and 40% carbohydrate (Karr-Lilienthala L. K et al, Livestock Production Science, 2005, Vol. 97:1-12). Soybean processing typically begins with dehulling, followed by crushing of the beans, and hexane extraction to isolate the soybean oil. Once the oil is extracted, the remaining material, composed primarily of protein and carbohydrate, is milled to produce commercial products such as soy grits and soy meal, which are primarily marketed and sold as animal feed. The carbohydrate component of those products constitutes most of the weight of the product. Conventionally, this carbohydrate component has a negative value. It can only be minimally digested by livestock, and cannot be digested at all by humans (Isao Hirose et al, Microbiology, 2000, 146 (Pt 1):65-75. http://mic.sgmjournals.org/cgi/content/full/146/1/65?view=long&pmid=10658653). Furthermore, the carbohydrate has been shown to cause gastrointestinal distress in livestock and in humans (Falkoski, D L et al., J. Agric. Food Chem., 2006, 54 (26):10184-10190). In addition, the consumption of the soy carbohydrate by monogastric livestock leads to increased production of methane, which is a serious greenhouse gas (Smiricky-Tjardes M R et al, J Anim. Sci., 2003, 81(10):2505-14. http://jas.fass.org/cgi/content/full/81/10/2505). Therefore, there is a great need for more efficient utilization of these soy components.

### SUMMARY OF THE INVENTION

The present invention provides microorganisms and methods that allow efficient utilization of soy components as carbon sources. In particular, the present invention provides engineered microorganisms that can efficiently convert soy carbohydrates to industrial chemicals by fermentation.

In one aspect, the present invention provides an engineered microbial cell comprising a modification that increases efficiency of utilization of a soy carbon source as compared with a parent cell. In some embodiments, a suitable soy carbon source is soy molasses, soy meal, soy hulls and/or an extract thereof. In some embodiments, a suitable soy carbon source is a cellulosic component present in the soy molasses, soy meal, soy hulls and/or the extract thereof. In some embodiments, a suitable cellulosic component is selected from the group consisting of cellulose, cellobiose, hemicellulose, pectin, verbascose, stachyose, raffinose, melibiose, xylose, xylan, lignin and combination thereof.

In some embodiments, a modification that increases efficiency of utilization of a soy carbon source includes altered (e.g., increased) expression or activity of a carbohydrate modifying enzyme. In some embodiments, the expression or activity of a carbohydrate modifying enzyme is increased by overexpression. In some embodiments, a modification that increases efficiency of utilization of a soy carbon source includes altered localization of a carbohydrate modifying enzyme. In some embodiments, a carbohydrate modifying enzyme according to the invention is modified to contain a secretory signal sequence.

In some embodiments, a carbohydrate modifying enzyme suitable for the invention is an enzyme naturally expressed by the microbial cell that is engineered. In some embodiments, a carbohydrate modifying enzyme is an enzyme that is not naturally expressed by the microbial cell that is engineered. In some embodiments, a suitable carbohydrate modifying enzyme is selected from the group consisting of melibiases, α-galactosidases, β-fructosidases, exoglucanases, acetyl esterases, α-glucuronidases, endoglucanases, cellobiohydrolases, xylanases, beta-xylosidases, alpha-L-arabinofuranosidases, acetyl xylan esterases, mannanases, xyloglucanases, polygalacturonases, exo-beta-1,3-glucosidases, lignin peroxidases, and combination thereof. In some embodiments, a suitable carbohydrate modifying enzyme includes an α-galactosidase encoded by RafA gene from E. coli. In some embodiments, a suitable carbohydrate modifying enzyme includes an exoglucanase selected from cellobiose hydrolase I and/or II. In some embodiments, a suitable carbohydrate modifying enzyme includes an endoglucanase selected from endoglucanase from T. reesei, endoglucanase I (EG I), EG II, EG III, or combination thereof. In some embodiments, a suitable carbohydrate modifying enzyme comprises cellobiohydrolase I (CBH I) and/or CBH II. In some embodiments, a suitable carbohydrate modifying enzyme comprises xylanase I (XYL I) and/or XYL II. In some embodiments, a suitable carbohydrate modifying enzyme comprises a lignin peroxidase produced by Phanerochaete chrysosporium.

In some embodiments, a modification that increases efficiency of utilization of a soy carbon source includes increased expression or activity of a saccharide transporter (e.g., a galactose importer).

In some embodiments, the present invention provides an engineered bacterial cell. In some embodiments, an engineered bacterial cell is selected from the group consisting of Bacillus, Clostridium, Enterobacter, Klebsiella, Micromonospora, Actinoplanes, Dactylosporangium, Streptomyces, Kitasatospora, Amycolatopsis, Saccharopolyspora, Saccharothrix, Actinosynnema and combination thereof. In particular embodiments, an engineered bacterial cell is a Bacillus cell (e.g., a Bacillus subtilis cell).

In some embodiments, an engineered cell produces a product of interest. In some embodiments, a product of interest is selected from the group consisting of a polypeptide, a non-ribosomal peptide, an acyl amino acid, a lipopeptide and combination thereof. In some embodiments, a product of interest comprises a lipopeptide. In some embodiments, the lipopeptide is a surfactin. In some embodiments, the lipopeptide is FA-Glu.

In another aspect, the present invention provides a fermentation process comprising growing an engineered microbial cell described herein in a culture medium comprising a soy carbon source (e.g., soy molasses, soy meal, soy hulls, an/or an extract thereof). In some embodiments, a medium used in the fermentation lacks a carbon source other than the soy carbon source. In some embodiments, the fermentation process is a submerged fermentation process. In some embodiments, the fermentation process is a solid state fermentation process. In some embodiments, the fermentation process converts at least 10% (e.g., at least 15%, 20%, 25%, 30%, 35%, 40%, 45%, 50%) of the soy carbon source into chemical products.

In yet another aspect, the present invention provides a method of producing an industrial chemical comprising growing an engineered microbial cell in a culture medium comprising a soy carbon source, wherein the engineered microbial cell comprises a modification that increases efficiency of utilization of the soy carbon source as compared with a parent cell, and further wherein the engineered microbial cell produces an industrial chemical of interest.

In some embodiments, a suitable soy carbon source comprises soy molasses, soy meal, soy hulls, an/or an extract thereof. In some embodiments, a suitable culture medium lacks a carbon source other than the soy carbon source. In some embodiments, the engineered microbial cell is an engineered Bacillus subtilis cell. In some embodiments, the industrial chemical of interest is selected from the group consisting of a polypeptide, a non-ribosomal peptide, an acyl amino acid, a lipopeptide and combination thereof. In some embodiments, the industrial chemical of interest comprises a lipopeptide. In some embodiments, the lipopeptide is a surfactin. In some embodiments, the lipopeptide is FA-Glu.

The details of one or more embodiments of the invention are set forth in the description below. Other features, objects, and advantages of the invention will be apparent from the description and from the claims. All cited patents, patent applications, and references (including references to public sequence database entries) are incorporated by reference in their entireties for all purposes.

### BRIEF DESCRIPTION OF THE DRAWINGS

The drawings are for illustration purposes only and not for limitation.

FIG. 1. Exemplary strategy in B. subtilis 168 for increasing efficiency of utilization of soy molasses. All relevant carbohydrate modifying enzymes are secreted into the external environment and the oligosaccharides are metabolized outside the cell. Monosaccharides are imported by transporters. *Melibiose may be produced by non-specific action of sucrase or other secreted enzymes on raffinose.

FIG. 2. Exemplary results illustrating robot-assisted selection of Bacillus subtilis candidates with successful chromosomal modifications.

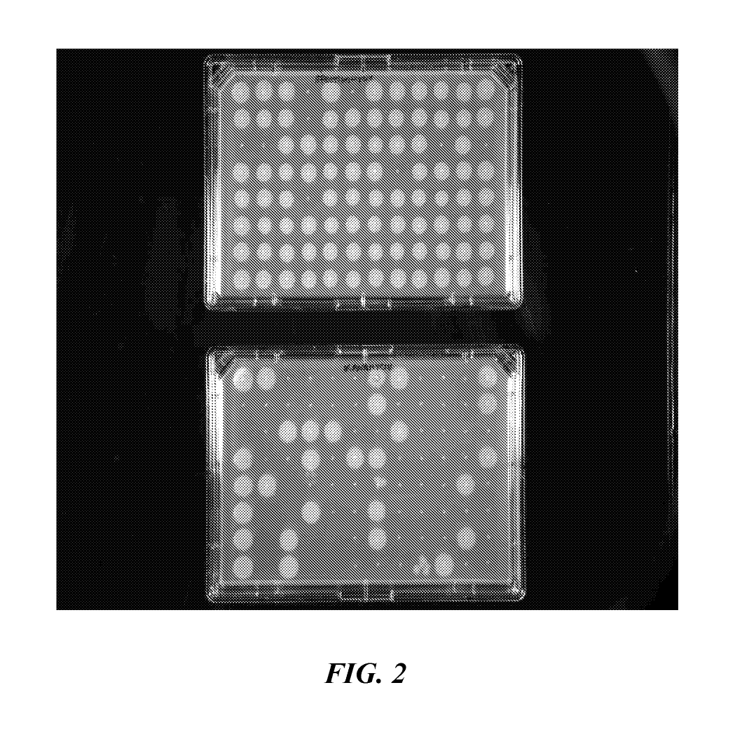

FIG. 3. Exemplary predominant oligosaccharides in Soy molasses (Qureshi N, Lolas A, Blaschek H P, J Microbiol Biotechnol., 2001, 26(5):290-5).

FIG. 4. Exemplary bacterial cyclic lipopeptide, Surfactin. Its structure includes a peptide loop of seven amino acids attached to a hydrophobic fatty acid chain thirteen to fifteen carbons long.

FIG. 5. Exemplary modular structure of surfactin synthetase. Each module consists of several domains with defined functions and is responsible for the addition of a single amino acid to the growing chain.

FIG. 6. Exemplary acyl amino acid. (a) Chemical structure of acyl amino acid with glutamate attached to a lipid moiety. (b) Modular structure of the modified surfactin synthetase operon. As compared to FIG. 5, modules 2-7 have been deleted.

FIG. 7. Exemplary surface tension profiles of Myristoyl Gluatmate and FA-Glu. FA-Glu Lipopeptide shows higher surface activity. CMC is about 1.3 mM. Data for FA-Glu (solid line). Data for myristoyl glutamate (dotted line).

FIG. 8. Exemplary strategy in B. subtilis 168 for increasing efficiency of utilization of soy molasses. This strategy involves supplementing it with a raffinose/stachyose-specific α-galactosidase (e.g., rafA from E. coli). A galactose importer, encoded by the gene galP, is also incorporated. *Melibiose may either be imported or produced by non-specific action of sucrase on raffinose.

### DEFINITIONS

In order for the present invention to be more readily understood, certain terms are first defined below. Additional definitions for the following terms and other terms are set forth throughout the specification.

“Acyl amino acid”: The term “acyl amino acid” as used herein refers to an amino acid that is covalently linked to a fatty acid. In certain embodiments, acyl amino acids are produced in microorganisms expressing engineered polypeptides, e.g., engineered polypeptides comprising a peptide synthetase domain covalently linked to a fatty acid linkage domain and a thioesterase domain or reductase domain. In certain embodiments, acyl amino acids are produced in microorganisms expressing engineered polypeptides comprising a peptide synthetase domain covalently linked to a beta-hydroxy fatty acid linkage domain and a thioesterase domain. In certain embodiments, acyl amino acids are produced in microorganisms expressing engineered polypeptides comprising a peptide synthetase domain covalently linked to a beta-hydroxy fatty acid linkage domain and a reductase domain. In certain embodiments, an acyl amino acid produced by a method described herein comprises a surfactant such as, without limitation, an acylated glutamate, e.g., cocoyl glutamate. In certain embodiments, acyl amino acids produced by compositions and methods of the present invention comprise a beta-hydroxy fatty acid. A beta-hydroxy fatty acid may contain 3, 4, 5, 6, 7, 8, 9, 10, 11, 12, 13, 14, 15, 16, 17, 18, 19, 20 or more carbon atoms. In some embodiments, a beta-hydroxy fatty acid is beta-hydroxy myristic acid, which contains 13 to 15 carbons in the fatty acid chain.

“Carbon source”: The term “carbon source” as used herein refers to a component of a cell culture medium that comprises carbon and that is utilized by a cell (e.g., a microbial cell) in culture medium for producing energy, cellular components, and/or metabolic products. Examples of carbon sources used in cell culture media include sugars, carbohydrates, organic acids, and alcohols (e.g., glucose, fructose, mannitol, starch, starch hydrolysate, cellulose hydrolysate, molasses, acetic acid, propionic acid, lactic acid, formic acid, malic acid, citric acid, fumaric acid, glycerol, inositol, mannitol and sorbitol). As used herein, the term “soy carbon source” refers to a carbon source derived from soy components, such as, soy molasses, soy meal, soy hulls and/or an extract thereof. See, definition of “soy components”.

“Cellulosic component”: As used herein, the term “cellulosic component” refers to any substance made from cellulose or a derivative of cellulose. An exemplary cellulose component can be, for example, cellulose, hemicellulose (e.g., xylan, xyloglucan, arabinoxylan, arabinogalactan, glucuronoxylan, glucomannan and galactomannan), pectin, xylan, lignin, C5 or C6 sugars derived from cellulose (e.g., verbascose, stachyose, raffinose, melibiose, xylose, cellobiose, fucose, and apiose), or combination thereof.

“Culture medium”: The term “culture medium” as used herein refers to any type of medium suitable for growth of a cell (e.g., a cell of a microorganism, e.g., a bacterial cell and/or a fungal cell). In some embodiments, a culture medium comprises medium in liquid form. In some embodiments, a culture medium comprises medium in solid form (e.g., solid agar).

“Lipopeptide”: The term “lipopeptide” as used herein refers to any of a variety of molecules that contain a peptide backbone covalently linked to one or more fatty acid chains. Often, lipopeptides are produced naturally by certain microorganisms. Lipopeptides can also be produced in microorganisms that are engineered to express the lipopeptides. A lipopeptide is typically produced by one or more nonribosomal peptide synthetases that build an amino acid chain without reliance on the canonical translation machinery. For example, surfactin is cyclic lipopeptide that is naturally produced by certain bacteria, including the Gram-positive endospore-forming bacteria Bacillus subtilis. Surfactin consists of a seven amino acid peptide loop, and a hydrophobic fatty acid chain (beta-hydroxy myristic acid) thirteen to fifteen carbons long. The fatty acid chain allows permits surfactin to penetrate cellular membranes. The peptide loop is composed of the amino acids glutamic acid, leucine, D-leucine, valine, aspartic acid, D-leucine and leucine. Glutamic acid and aspartic acid residues at positions 1 and 5 respectively, constitute a minor polar domain. On the opposite side, valine residue at position 4 extends down facing the fatty acid chain, making up a major hydrophobic domain. Surfactin is synthesized by the linear nonribosomal peptide synthetase, surfactin synthetase is synthesized by the three surfactin synthetase subunits SrfA-A, SrfA-B, and SrfA-C. Each of the enzymes SrfA-A and SrfA-B consist of three amino acid activating modules, while the monomodular subunit SrfA-C adds the last amino acid residue to the heptapeptide. Additionally the SrfA-C subunit includes the thioesterase domain (“TE domain”), which catalyzes the release of the product via a nucleophilic attack of the beta-hydroxy of the fatty acid on the carbonyl of the C-terminal Leu of the peptide, cyclizing the molecule via formation of an ester. Other lipopeptides and their amino acid and fatty acid compositions are known in the art, and can be produced in accordance with compositions and/or methods of the present invention. In certain embodiments, lipopeptides are produced by a method described herein in microorganisms engineered to express one or more polypeptides that participate in lipopeptide synthesis. In certain embodiments, lipopeptides produced by compositions and methods of the present invention comprise a beta-hydroxy fatty acid. A beta-hydroxy fatty acid may contain 3, 4, 5, 6, 7, 8, 9, 10, 11, 12, 13, 14, 15, 16, 17, 18, 19, 20 or more carbon atoms. In some embodiments, a beta-hydroxy fatty acid is beta-hydroxy myristic acid, which contains 13 to 15 carbons in the fatty acid chain.

“Nitrogen source”: The term “nitrogen source” as used herein refers to a component of a cell culture medium that comprises nitrogen and is utilized by a cell (e.g., a microbial cell) in culture medium for growth. Examples of nitrogen sources include soy extract, tryptone, yeast extract, casamino acids, distiller grains, ammonia and ammonium salts (e.g., ammonium chloride, ammonium nitrate, ammonium phosphate, ammonium sulfate, ammonium acetate), urea, nitrate, nitrate salts, amino acids, fish meal, peptone, corn steep liquor, and the like.

“Non-ribosomal peptide”: The term “non-ribosomal peptide” as used herein refers to a peptide chain produced by one or more nonribosomal peptide synthetases. Thus, as opposed to “polypeptides” (see definition, infra), non-ribosomal peptides are not produced by a cell's ribosomal translation machinery. Polypeptides produced by such nonribosomal peptide synthetases may be linear, cyclic or branched. Numerous examples of non-ribosomal peptides that are produced by one or more nonribosomal peptide synthetases are known in the art. One non-limiting example of non-ribosomal peptides that can be produced in accordance with the present invention is surfactin. Those of ordinary skill in the art will be aware of other non-ribosomal peptides that can be produced using compositions and methods of the present invention. In certain embodiments, a non-ribosomal peptide contains one or more covalently-linked fatty acid chains and is referred to herein as a lipopeptide (see definition of “lipopeptide”, supra).

“Polypeptide”: The term “polypeptide” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified. For example, a polypeptide may be glycosylated. A polypeptide can comprise two or more polypeptides that function as a single active unit.

“Soy components”: As used herein, “soy components” include any type of compositions produced by and/or derived from, soybeans (e.g., any type of composition produced from any part of a soybean). Soy components used as a carbon source for cell culture include carbohydrates. In some embodiments, soy components used as a carbon source for cell culture comprise soy molasses, soy meal, soy hulls and/or an extract thereof.

“Soy molasses”: Soy molasses, as used herein, refers to an extract of soybeans which is rich in carbohydrates. In some embodiments, soy molasses is an alcohol extract of soybeans. In some embodiments, soy molasses is produced by aqueous alcohol extraction of defatted soybean material (e.g., defatted soybeans). In some embodiments, soy molasses is produced by extracting soybean material with an aqueous alcohol, such as aqueous ethanol, aqueous isopropanol or aqueous methanol, and by removing alcohol from the extract. In some embodiments, soy molasses contains 10%, 20%, 30%, 40%, 50%, 60%, or 70% total soluble solids. In some embodiments, soy molasses used in a composition or method described herein is sterilized (e.g., by autoclaving).

“Soy hulls”: The term “soy hulls” as used herein refers to a soybean by-product that primarily contain the skin of the soybean which comes off during dehulling processing. Soy hulls as used herein include both processed and unprocessed soy hulls. In some embodiments, processed soy hulls are treated with enzymes such as cellulase, beta-glucosidase, hemicellulase and/or pectinase.

“Soy meal”: The term “soy meal” as used herein refers to a soybean by-product typically obtained by grinding the flakes which remain after removal of most of the oil from soybeans by a solvent or mechanical extraction process.

“Substantially”: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

“Substantially lacks”: The term “substantially lacks” as used herein refers to the qualitative condition of exhibiting total or near-total absence of a particular component. One of ordinary skill in the biological arts will understand that biological and chemical compositions are rarely, if ever, 100% pure. Conversely, one of ordinary skill in the biological arts will understand that biological and chemical compositions are rarely, if ever, 100% free of a particular component. The term “substantially lacks” is therefore used herein to capture the concept that a biological and chemical composition may comprise a small, inconsequential amount of one or more impurities. To give but one particular example, when it is said that a cell culture medium “substantially lacks” a given component, it is meant to indicate that although a minute amount of that component may be present (for example, as a result of being an impurity and/or a breakdown product of one or more components of the cell culture medium, or as a result of being a minor component of a pre-seed culture which is inoculated into a seed or production culture), that component is nevertheless an inconsequential part of the cell culture medium and does not alter the basic properties of that cell culture medium. In certain embodiments, the term “substantially lacks”, as applied to a given component of a cell culture medium, refers to condition wherein the cell culture medium comprises less that 5%, 4%, 3%, 2%, 1%, 0.9%, 0.8%, 0.7%, 0.6%, 0.5%, 0.4%, 0.3%, 0.2%, 0.1% or less of that component. In certain embodiments, the term “substantially lacks”, as applied to a given component of a cell culture medium, refers to condition wherein the cell culture medium lacks any detectable amount of that component.

### DETAILED DESCRIPTION OF CERTAIN EMBODIMENTS

The present invention provides, among other things, engineered microorganisms and methods that allow efficient conversion of soy carbohydrates to industrial chemicals by fermentation. In some embodiments, the invention provides microbial cells engineered to have increased efficiency in utilizing a soy carbon source (e.g., soy molasses, soy meal, and/or soy hulls). In some embodiments, microbial cells are engineered to have altered (e.g., increased) expression or activity of one or more carbohydrate modifying enzymes (e.g., glycosidases). In some embodiments, microbial cells are engineered to have altered localization of carbohydrate modifying enzymes (e.g., glycosidases). In some embodiments, engineered microbial cells provided herein are used to produce industrial chemicals (e.g., surfactin) using soy components as primary or sole carbon sources.

Among other things, inventive methods and compositions provided herein give commercial-scale soybean processors an incentive to use established methods (i.e., alcohol precipitation) to separate soy protein from soy carbohydrate. The isolated soy protein will be a superior product for use in food and feed, and the value of the carbohydrate fraction will be increased as it can be used as a feedstock for production of industrial chemicals by fermentation according to the present invention. Therefore, the present invention will bring a fundamental change in the nature of soybean processing which will have a significant impact on our economy. According to the USDA-NASS, the United States produced 80 million metric tons of soybeans in 2008 (USDA—National Agricultural Statistical Service Iowa Field Office, Agri-News, 2008, Vol 8-18). Given that 40% of the mass of a soybean is carbohydrate, the U.S. produced 70 billion pounds of soy carbohydrate in 2008. A fermentation process that can convert 50% of that material to chemicals products will produce 35 billion pounds of “green chemicals” from this waste material annually.

For example, inventive methods and compositions described herein can be used for conversion of soy carbohydrate to useful products such as specialty surfactants. As described below, by cleaving the glycosidic bonds in the soy carbohydrates (e.g., galacto-oligosaccharides) enzymatically, simple sugars become available and can be utilized as a carbon source to support cell growth and surfactant production. Thus, engineered strains provided by the present invention enable cost effective production of surfactants and other chemicals using soy components (e.g., soy molasses and/or soy hulls) as inexpensive feedstock. According to a report prepared by the consulting firm OmniTech for the United States Soybean Board, current annual U.S. production of specialty surfactants is about 1 million tons. Thus, the U.S. generates 16 times more soy carbohydrate than what is needed to produce our nations entire annual output of specialty surfactants. Moreover, the present invention has uses beyond production of surfactants.

The present invention will also bring a significant impact on our environment. Currently, only 5% of all chemical products are made from renewable materials (Bachmann, R., Riese, J., Value Creation, Eds. Budde F., Felcht U H., Frankemölle H, 2^{nd} Edition, Wiley-V C H, 2006: 375-388). Cargill has estimated that about 65% of our chemicals can be made from renewable materials (Wedin, R., Chemistry, 2004:30-35. http://www.wedincommunications.com/ChemistryHighCarb.pdf). The consulting firm McKinsey and Company has estimated that if we can increase the fraction of chemicals produced using renewable material from 5% to 20%, that switch alone will enable us to achieve 20% of the carbon dioxide reduction goals of the Kyoto protocol (Bachmann, R., Riese, J., Value Creation, Eds. Budde F., Felcht U H., Frankemölle H, 2^{nd} Edition, Wiley-VCH, 2006: 375-388). Furthermore, this approach will have a second environmental benefit. The surfactants produced according to the present invention are readily biodegradable.

Various aspects of the invention are described in detail in the following sections. The use of sections is not meant to limit the invention. Each section can apply to any aspect of the invention. In this application, the use of “or” means “and/or” unless stated otherwise.

### Engineering Microbial Cells

Any of a variety of microorganisms can be engineered as described herein and may be grown on a soy carbon source according to the present invention. As non-limiting examples, bacteria of the genera Bacillus, Clostridium, Enterobacter, Klebsiella, Micromonospora, Actinoplanes, Dactylosporangium, Streptomyces, Kitasatospora, Amycolatopsis, Saccharopolyspora, Saccharothrix and Actinosynnema may be grown in accordance with compositions and/or methods of the present disclosure. In certain embodiments, a bacterium of the genus Bacillus is engineered according to the present invention. In certain embodiments, a bacterium of the species Bacillus subtilis is engineered according to the present invention.

In some embodiments, microbial cells are engineered to increase efficiency of utilization of a soy carbon source as compared with a parent cell. As used herein, a soy carbon source refers to a carbon source used in a cell culture medium that is substantially or solely composed of soy components, such as, soy molasses, soy meal, soy hulls and/or extracts thereof. As used herein, a soy carbon source is the sole carbon source in a cell culture medium if the cell culture medium substantially lacks other carbon sources. In some embodiments, a soy carbon source is a cellulosic component present in the soy molasses, soy meal, soy hulls or extracts thereof. Examples of cellulosic components include, but are not limited to, cellulose, cellobiose, hemicellulose, pectin, xylan, lignin, and various saccharides and C5, C6 sugars resulting from decomposition of cellulosic materials such as verbascose, stachyose, raffinose, melibiose, xylose, and combination thereof. As used herein, the efficiency of utilization of a carbon source can be measured using various methods known in the art. In some embodiments, the efficiency of utilization of a carbon source can be measured using volumetric productivity. Typically, volumetric productivity indicates a relation of the output and the time requirement in a reacting system, e.g., fermentation bioreactor. In some embodiments, volumetric productivity is measured by the amount of a chemical product of interest produced per liter of soy component per day under a pre-determined condition. In some embodiments, a chemical product of interest is a surfactant (e.g., surfactin). In particular embodiments, a chemical product of interest is FA-Glu (fatty acid-glutamate). In some embodiments, engineered microbial cells according to the present invention increase the volumetric productivity of a chemical product of interest (e.g., FA-Glu) by at least 10%, 15%, 20%, 25%, 30%, 35%, 40%, 45%, 50%, 60%, 65%, 70%, 75%, 80%, 90%, 95%, as compared to a parent cell. In some embodiments, engineered microbial cells according to the present invention increase the volumetric productivity of a chemical product of interest (e.g., FA-Glu) by at least 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold as compared to a parent cell.

In some embodiments, microbial cells are engineered to contain a modification that increases efficiency of utilization of a soy carbon source. In some embodiments, microbial cells are engineered to contain altered (e.g., increased) expression or activity of a carbohydrate modifying enzyme. In some embodiments, microbial cells are engineered to overexpress a carbohydrate modifying enzyme. In some embodiments, microorganisms are engineered such that carbohydrate modifying enzymes (e.g., glycosidases such as, for example, meliabiase, α-galactosidase, β-fructosidase, or a combination thereof) have altered localization. For example, microorganisms can be modified to secrete glycosidases that are not naturally secreted. Such modifications can include addition of a secretory signal to sequences encoding the carbohydrate modifying enzyme.

Carbohydrate Modifying Enzymes

Various carbohydrate modifying enzymes can be used in the present invention, in particular, those enzymes (e.g., glycosidases) that can break down carbohydrates present in soy components (e.g., soy molasses, soy meal and/or soy hulls). Exemplary carbohydrate modifying enzyme suitable for the present invention include, but are not limited to, melibiases, α-galactosidases, β-fructosidases, exoglucanases, acetyl esterases, α-glucuronidases, endoglucanases, cellobiohydrolases, xylanases, beta-xylosidases, alpha-L-arabinofuranosidases, acetyl xylan esterases, mannanases, xyloglucanases, polygalacturonases, exo-beta-1,3-glucosidases, lignin peroxidases, and combination thereof.

Specific non-limiting examples of suitable enzymes include, but are not limited to, melibiase enzyme of Bacillus subtilis, encoded by the melA gene, useful to cleave the galactose-glucose linkages in melibiose, stachyose and raffinose; RafA gene from E. coli encoding an α-galactosidase that allows for utilization of raffinose and stachyose present in soy molasses; exoglucanases such as cellobiose hydrolases I and II; cellobiohydrolases (1,4-β-D-glucan cellobiohydrolase, EC 3.2.1.91); endoglucanases such as endo-1,4-β-glucanases, EC 3.2.1.4), and endoglucanase I from T. reesei; exoglucohydrolase (1,4-β-D-glucan glucohydrolase, EC 3.2.1.74); β-glucosidases such as that from Aspergillus niger; endo-1,4-α-xylanases (A and D); α-L-arabinofuranosidases; acetyl esterases; α-glucuronidases; endoglucanase I (EG I); EG II; EG III; cellobiohydrolase I (CBH I); CBH II; xylanase I (XYL I), xylanase II (XYL II), beta-xylosidase, alpha-L-arabinofuranosidase, acetyl xylan esterase, mannanase, alpha-galactosidase, xyloglucanase, polygalacturonase, exo-beta-1,3-glucosidase, endoxylanases, acetylesterases (EC 3.1.1.6), α-L-arabinofuranosidase (EC 3.2.1.55), α-glucuronidase (EC 3.2.1.139), β-xylosidases (EC 3.2.1.37), and lignin peroxidase.

One or more enzymes described herein can be overexpressed in a microbial cell. In some embodiments, a microbial cell is engineered to overexpress a carbohydrate modifying enzyme naturally expressed by the cell. In some embodiments, a microbial cell is engineered to overexpress a carbohydrate modifying enzyme that is not naturally expressed by the cell. For example, multiple variants of an enzyme can be used, e.g., α-galactosidase and/or β-fructosidase enzyme variants from multiple Rhizopus species, which have a strong ability to hydrolyze the glycosidic bonds in soy bean oligosaccharides (Rehms, H., Barz W., Appl Microbiol Biotechnol, 1995, 44: 47-52). In some embodiments, a microbial cell is engineered to overexpress one or more carbohydrate modifying enzymes that supplement the enzymes already present in the cell to facilitate break down of carbohydrates. For example, endoglucanases hydrolyse amorphous regions of the cellulose fibres. The non-reducing ends generated could be attacked by exoglucanases, which proceed with the degradation of crystalline regions. β-Glucosidases hydrolyse cellobiose, which prevents the inhibition of cellobiohydrolase by this disaccharide. As non-limiting example, Bacillus cells have a putative endoglucanase, an endo-1,4-β^{˜}-glucanase, and another putative endo-1,4-β^{˜}-glucanase to break cellulose. However, it lacks exoglucanases. Therefore, a Bacillus cell can be engineered to overexpress, e.g., cellobiose hydrolases I and II from T. reesei, as well as β-glucosidase from Aspergillusniger. If the endo-glucanases present in a Bacillus strain are not very active, the cells can be engineered to also overexpress endoglucanase I from T. reesei.

As another non-limiting example, microbial cells are engineered to break down hemicelluloses. Hemicelluloses are complex heteropolysaccharides. Xylan is the major component of hemicellulose, whose abundance ranges between 20-24% of all sugars. The backbone of xylan is a polymer of β-1,4-linked D-xylosyl residues, which are substituted with arabinosyl, acetyl and glucuronosyl residues. The frequency and composition of the branches are dependent on the source of the xylan. The degradation of xylan requires a large number of different enzymes. The xylan backbone is degraded by endo-β-1,4-xylanases (EC 3.2.1.8). However, endoxylanases are often prevented from cleaving the xylan backbone by the presence of the above mentioned substituents. Typically, these substituents need to be removed before endoxylanase can efficiently hydrolyse the backbone. The enzymes involved include acetylesterases (EC 3.1.1.6), α-L-arabinofuranosidase (EC 3.2.1.55), and α-glucuronidase (EC 3.2.1.139). Once endoxylanases have released small xylooligosaccharides, the β-xylosidases (EC 3.2.1.37) cleave the oligomeric fragments, predominantly to xylose. Bacillus strain has two endo-1,4-α-xylanases (A and D) as well as two α-L-arabinofuranosidase. However, it lacks an acetyl esterase and a α-glucuronidase, both of which are important to degrade hemicellulose. Thus, in some embodiments, a Bacillus cell is engineered to express both of these enzymes obtained from T. reesei. In some embodiments, a Bacillus cell is engineered to express all enzymes involved in T. reesei cellulose biodegradation including endoglucanase I (EG I), EG II, EG III, cellobiohydrolase I (CBH I), CBH II, xylanase I (XYL I), xylanase II (XYL II), beta-xylosidase, alpha-L-arabinofuranosidase, acetyl xylan esterase, mannanase, alpha-galactosidase, xyloglucanase, polygalacturonase, and exo-beta-1,3-glucosidase. An alternative organism for these enzymes is A. niger.

As yet another non-limiting example, microbial cells are engineered to degrade lignin. Lignin is an aromatic polymer, consisting of a variety of structurally related phenylpropanoid subunits, which are typically linked via ether or direct C—C bonds. Lignin is highly resistant to biodegradation, which is assumed to occur only in the presence of molecular oxygen with the aid of peroxidases and oxidases. Bacillus strain typically lacks the appropriate enzymes to biodegrade lignin. In some embodiments, a Bacillus strain is engineered to express lignin peroxidase produced by a fungi such as Phanerochaetechrysosporium. In some embodiments, a chemical/physical (alkaline) process is used during fermentation to degrade lignin.

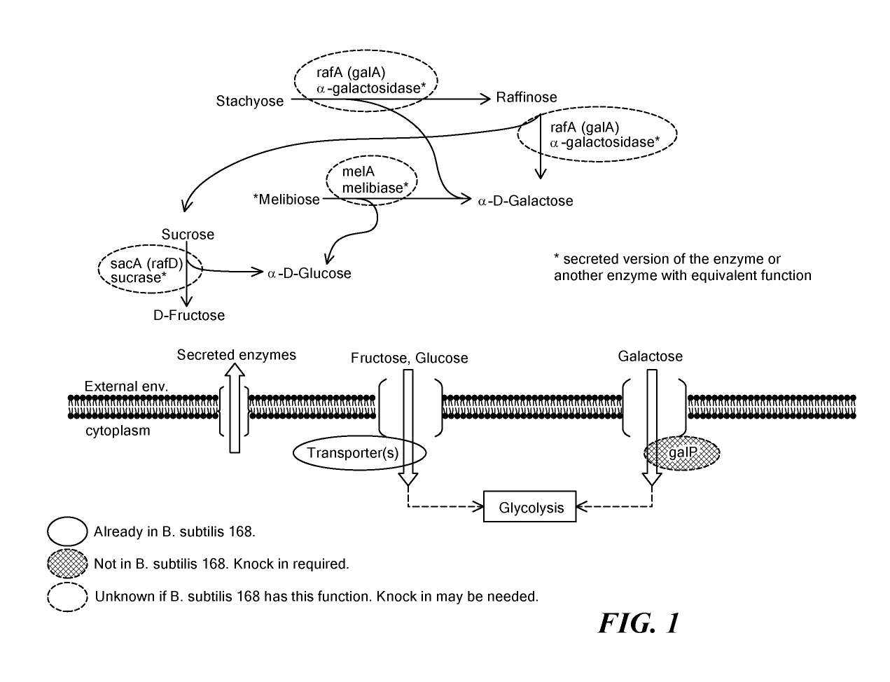

In some embodiments, microbial cells are engineered to have altered localization of a carbohydrate modifying enzyme. For example, genes encoding carbohydrate modifying enzymes (e.g., α-galactosidase and/or β-fructosidase) can be modified to add a secretory signal at the N-terminus of the proteins (see FIG. 1) resulting in secretion of the enzymes that modify soy carbohydrates (e.g., oligosaccharides) present in soy molasses, soy meal or soy hulls. Various secretory signal sequences are known in the art and can be used to practice the present invention. For example, secretory signal sequences found in proteins secreted by Bacillus cells can be used in the present invention. Without wishing to be bound by theory, it is contemplated that this type of strategy can be advantageous because the enzymes are secreted and act outside the cell and therefore will be less likely to cause regulatory effects within the cell such as catabolite repression due to changes in sugar levels. In some such embodiments, microbial cells are also engineered to express a saccharide transporter (e.g., a galactose importer). For example, a galactose importer, encoded by the gene galP from Lactobacillus brevis can be incorporated into Bacillus cells to enable import of any extracellular-galactose.

Other Modifications

In some embodiments, additional modifications may be introduced into a microbial cell to facilitate the utilization of a soy carbon source. Such additional modifications include enhanced importation of certain saccharides. For example, certain microbial strains such as Bacillus subtilis have all of the enzymes required to metabolize galactose (which is a major component of the galacto-oligosaccharides). However, wild type Bacillus subtilis strains are unable to transport galactose into the cell (Stülke J, Hillen W, Annu Rev Microbiol., 2000, 54:849-80). Therefore, Bacillus cells may be engineered to express a galactose importer such as, for example, a galactose importer encoded by the gene galP from Lactobacillus brevis, or ABC transporters encoded by the MsmEFGK operon genes from Streptococcus mutans.

In some embodiments, a microbial cell can be engineered to prevent the formation of certain carbohydrates that are difficult to be utilized by the cell as a carbon source. For example, Bacillus subtilis is known to secrete an enzyme (levansucrase) that transfers fructose from molecules such as sucrose or raffinose onto the fructose residue of an “acceptor molecule” (such as sucrose or raffinose). Repeated cycles of this process create a polymer composed mostly of fructose, but with a “starter unit” composed of sucrose or raffinose. The polymer is referred to as levan (Fujita Y., Biosci Biotechnol Biochem., 2009, 73(2):245-59. http://www.jstage.jst.go.jp/article/bbb/73/2/245/_pdf). Bacillus is able to utilize the levan as a carbohydrate source by secreting levanase, an enzyme that degrades the levan to yield fructose. This process, though, happens only when other carbon sources have been used up. It is contemplated that preventing the formation of levan may increase efficiency of carbohydrate utilization. Thus, in some embodiments, a Bacillus subtilis cell is engineered to have a deficiency (e.g., deletion) of a gene encoding a levansucrase.

In some embodiments, microbial cells may be engineered to incorporate one or more modifications described herein. For example, one strategy may involve optimizing import of the galacto-oligosaccharides into Bacillus, followed by optimization of utilization of the imported carbohydrates by overexpression of an α-galactosidase that is known to cleave raffinose and stacyose efficiently. An alternative strategy involves optimization of extracellular breakdown of the galacto-oligosaccharides and/or engineering aimed at optimizing uptake and utilization of the free sugars. These approaches can be used alone or in combination.

Methods of Engineering

Enzymes suitable for the invention include naturally-occurring enzymes or modified enzymes with amino acid sequence substitutions, deletions, insertions. Typically, a modified enzyme retains substantially the same catalytic activity as compared to the corresponding naturally-occurring enzyme. In some embodiments, a modified enzyme has enhanced catalytic activity as compared to the corresponding naturally-occurring enzyme. Enzymes may be cloned and incorporated into a microbial cell using standard recombinant technology. In some embodiments, an enzyme is under the control of a constitutive promoter so that the bacteria can use it during the entire growth phase. In some embodiments, an enzyme is under the control of an inducible promoter so that the enzyme can be induced at a desired stage.

In some embodiments, microbial cell engineering can take place at plasmid level. For example, desired enzymes may be cloned into suitable plasmids and transformed into a microbial cell of interest. In some embodiments, microbial cell engineering may take place at the chromosome level, especially, for those microbial strains (e.g., Bacillus) in which plasmids are not stable. In some embodiments, high throughput engineering of the chromosome is used to engineering a microbial cell of interest. For example, high throughput engineering of the Bacillus chromosome is used to produce an engineered Bacillus. Bacillus subtilis is GRAS (generally regarded as safe), and is widely used for industrial-scale production of chemicals by fermentation (Priesr F G., Fermentation process development of industrial organisms, Ed. Justin O. Neway, Marcel Dekker, 1989, 73-117 and Schallmey M, Singh A, Ward O P, Can J. Microbiol., 2004, 50(1):1-17). In addition, Bacillus is a well established organism for gene engineering (Doi R H, Biotechnol Genet Eng Rev., 1984, 2:121-55. and Rapoport G, Klier A., Curr Opin Biotechnol., 1990, 1(1):21-7). However, plasmids tend to be unstable in Bacillus (Bron S. et al., Res Microbiol., 1991, 142(7-8):875-83) which reduce the speed and efficiency of gene engineering in Bacillus. Thus, it is desirable to have gene engineering done at the chromosome level. Methods for chromosome engineering have been established for Bacillus (e.g., congression) but they typically require the screening of about 10,000 bacterial colonies in order to find a strain that harbors a particular desired genetic change (Dubnau, D., Biochemistry, Physiology, and Molecular Genetics, Eds Sonenshein, A. L., Hoch, J. A., and Losick, R., American Society for Microbiology, 1993:555-584). In order to overcome these limitations, the present inventors developed an automated process that enables rapid introduction of changes into the Bacillus chromosome (Fabret C., Ehrlich S D., Noirot P., Mol. Microbiol., 2002, 46: 25-36. http://www3.interscience.wiley.com/cgi-bin/fulltext/118923511/HTMLSTART and Jarrell. K A. et al., International Patent Application PCT/US2008/060474, Publication Number WO2008131002, 2008). With this approach any desired change can be made rapidly, including single base substitutions, deletions, or the building of large gene sets in the Bacillus chromosome.

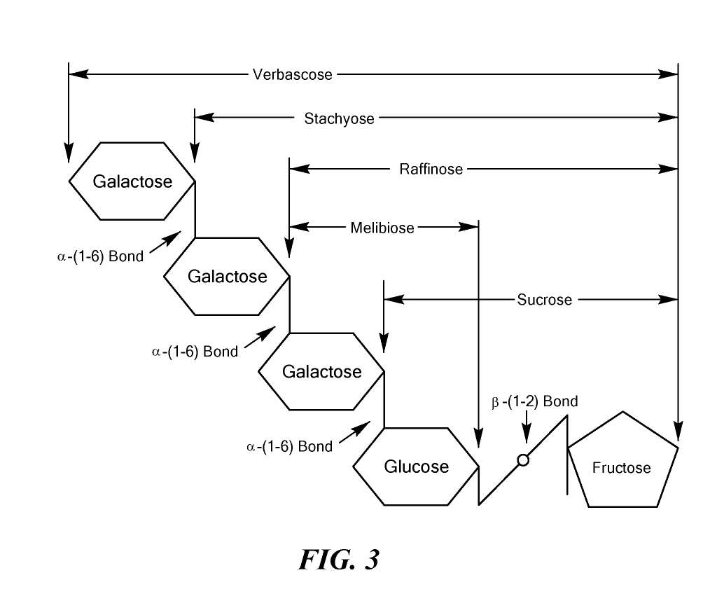

Data from a typical gene engineering experiment are shown in FIG. 3. The upper and lower plates differentiated in antibiotic selection. Precursor strains had Kanamycin resistance and new potentially engineered strains of interest are Kanamycin sensitive and therefore do not grow on the lower plate. The upper plate shows 88 colonies that we isolated from a gene engineering experiment. Strains that grow on the upper plate but fail to grow on the lower plate are likely to harbor a desired gene engineering event. Note that 56 colonies grew on the upper plate but failed to grow on the lower plate. In this particular experiment, the goal was to simultaneously produce 28 engineered strains, each of which harbors a particular deletion. The 56 colonies that met the selection criteria were characterized using PCR followed by DNA sequencing. All 28 of the desired deletion strains were identified upon sequencing of only 56 colonies. Using convention methods, such as congression, it would have been necessary to screen 280,000 colonies in order to achieve this same result.

### Soy Carbon Sources

Soy components, e.g., low cost soy components such as soy molasses, soy hulls, and/or soy meal, can be used as a primary or sole carbon source for the growth of engineered microorganisms provided herein.

Soy Molasses

Soy molasses is made up of multiple carbohydrates. Typically, the carbohydrate composition of which varies from batch to batch. Carbohydrates in soy molasses include mono- and disaccharides like dextrose, sucrose and fructose and also oligosaccharides such as raffinose, stachyose and verbascose. These three oligosaccharides are composed of Galactose, Glucose and Fructose subunits linked by α-1-6 and β-1-2 glycosidic bonds (FIG. 3) and are often referred to as “galacto-oligosaccharides”.

In certain embodiments, soy molasses is an industrial aqueous alcohol extract of soybeans, usually produced as a residual by-product during the production of soybean protein isolates and concentrates. In some embodiments, soy molasses is produced by aqueous alcohol extraction of defatted soybean material, such as defatted soybean flakes, with a warm aqueous alcohol, such as aqueous ethanol, aqueous isopropanol or aqueous methanol. Thereafter the alcohol and some of the water, as is desired, are removed by methods such as evaporation, distillation, steam stripping, to obtain a substantially alcohol free soy molasses with a desired moisture content.

In some embodiments, soy molasses contains 20%, 30%, 40%, 50%, 60%, or 70% total soluble solids. The solids typically include carbohydrates, proteins and other nitrogenous substances, minerals, fats and lipoids. The major constituents of soy molasses are sugars that include oligosaccharide (stachyose and raffinose), disaccharides (sucrose) and minor amounts of monosaccharides (fructose and glucose). Minor constituents include saponins, protein, lipid, minerals (ash), isoflavones, and other organic materials. In certain embodiments, a cell culture medium includes soy molasses at a final concentration of about 0.1%, 0.5%, 1%, 2%, 3%, 4%, 5%, 6%, 7%, 8%, 9%, 10%, 11%, 12%, 13%, 14%, or 15% solids. For example, a soy molasses containing 50% solids can be added to a cell culture medium at a dilution of 1:50, 1:25, 1:16, 1:12.5, 1:10, etc.

Soy Hulls

Soy hulls, and carbohydrate compositions produced from soy hulls, are additional inexpensive feedstocks. Soy hulls may be provided in an unprocessed form, in a decomposed form, and/or in a form enriched for a particular cellulosic component, such as xylose, cellobiose, or xylan. In some embodiments, soy hulls are treated to release carbohydrates. Exemplary treatments for cellulosic raw materials include chemical (e.g., dilute acid, aqueous alkali treatment), mechanical, heat, and/or enzyme treatments. Dilute acid pretreatment is described in Grethlein, Bio/Technology 2:155-160, 1985; Schell et al., Appl. Biochem. Biotechnol. 77-79:67-81, 1999; and Torget, et al., Ind. Eng. Chem. Res. 39:2817-2825, 2000. Steam explosion treatment is described, e.g., in Brownell and Saddler, Biotechnol. Bioeng. 29:228-235, 1987; Heitz et al., Biores. Technol. 35:23-32, 1991; and Puls et al., Appl. Microbiol. Biotechnol. 22:416-423; 1985. Hydrothermal treatment is described, e.g., in Bobleter, Prog. Polym. Sci. 19:797-841, 1994; Laser et al., Biores. Technol. 81:33-44, 2002; and Mok and Antal. Ind. Eng. Chem. Res. 31:1157-1161, 1992. Organic solvent extraction is described, e.g., in Chum et al., Biotechnol. Bioeng. 31:643-649, 1988 and Holtzapple and Humphrey, Biotechnol. Bioeng. 26:670-676, 1984. Ammonia fiber explosion is described in Dale and Moriera, Biotechnol. Bioeng. Symp. Ser. 12:31-43, 1982. Sodium hydroxide treatment is described, e.g., in Weil et al., Enzyme Microb. Technol. 16:1002-1004, 1994. Lime treatment is described, e.g., in Chang et al., Appl. Biochem. Biotechnol. 63-65:3-19, 1997; and Kaar and Holtzapple, Biomass Bioenerg. 18:189-199, 2000. See also Wyman, Bioresour. Tech. 96(18):1959-66, 2005.

Soy hulls can be treated to release carbohydrates prior to or during use in a culture medium. In some embodiments, soy hulls are treated in a culture medium (e.g., soy hulls are provided in a culture medium with one or more enzymes that break down cellulosic material, e.g., cellulase, cellobiase, hemicellulase, and/or pectinase). In some embodiments, soy hulls are used which have not been treated to release carbohydrates. A culture medium can include soy hulls, or a component thereof, at a weight to volume ratio of 1, 2, 3, 4, 5, 6, 7, 8, 9, 10, 11, 12, 13, 14, 15%, or greater.

Soy Meal

Soy meal typically refers to a soybean by-product typically obtained by grinding the flakes which remain after removal of most of the oil from soybeans by a solvent or mechanical extraction process. Soy meal is a high quality protein filler containing about 50% protein. It is typically used as inexpensive pet food and boosts the protein content of the food.

Typically, soy meal is autoclaved in distilled H2O before use in fermentations. In some cultures, solid materials remained throughout fermentation. In other cultures, a Soy Meal Extract was used as the soy source. To make this extract, soy meal can be autoclaved at a higher concentration (i.e., 8%) and liquid soluble portion was removed, re-autoclaved and diluted to desired concentration in liquid media (i.e., 0.5%).

In certain embodiments, engineered microorganisms are grown in cell culture media that contain soy components (e.g., soy molasses, soy meal, and/or soy hulls) as a carbon source, which cell culture media further substantially lack an additional carbon source (e.g., the media lack added glucose and glycerol). In certain embodiments, microorganisms are grown in cell culture media that contain soy components (e.g., soy molasses, soy meal, and/or soy hulls) as the sole carbon source.

In certain embodiments, a cell culture medium includes soy molasses, soy meal and/or soy hulls at a final concentration of about 1%, 2%, 3%, 4%, 5%, 6%, 7%, 8%, 9%, 10%, 11%, 12%, 13%, 14%, 15%, 20%, 25%, or 30% solids. For example, a soy molasses containing 50% solids can be added to a cell culture medium at a dilution of 1:50, 1:25, 1:16, 1:12.5, 1:10, etc.

In certain embodiments, a medium including soy components is a medium for growing Bacillus in which a carbon source such as glucose is substituted with soy components (e.g., soy molasses, soy meal and/or soy hulls). In certain embodiments, a medium including soy components is a modified form of a medium described by Spizizen, Proc. Nat. Acad. Sci. USA 44(10):1072-0178, 1958. In certain embodiments, a medium including soy components includes the following: (NH₄)₂SO₄, K₂HPO₄, KH₂PO₄, Na₃-citrate dehydrate, magnesium sulfate heptahydrate, CaCl₂ dihydrate, FeSO₄ heptahydrate, disodium EDTA dihydrate, and soy molasses at 0.1%, 0.5%, 1%, 2%, 3%, 4%, 5%, 6%, 7%, 8%, 9%, or 10% solids. In certain embodiments, a medium including soy components includes the following: (NH₄)₂SO₄ at 2 g/L, K₂HPO₄ at 14 g/L, KH₂PO₄ at 6 g/L, Na₃-citrate dihydrate at 1 g/L, magnesium sulfate heptahydrate at 0.2 g/L, CaCl₂ dihydrate at 14.7 mg/L, FeSO₄ heptahydrate at 1.1 mg/L, disodium EDTA dihydrate at 1.5 mg/L, and soy molasses at 0.1%, 0.5%, 1%, 2%, 3%, 4%, 5%, 6%, 7%, 8%, 9%, or 10% solids. Additional exemplary media formulae are described in the Examples section. Further examples are described in U.S. Application Pub. Nos. 20100093060 and 20100093037, the disclosures of which are hereby incorporated by references. Other media formulae suitable for growing various microbial cells such as Bacillus are known and may be modified to include soy components as a carbon source in accordance with the present disclosure.

Various culture media containing soy carbon sources described herein can be used in various fermentation process. As used herein, the term “fermentation” refers to a process of conversion of carbohydrates into alcohols or acids. In some embodiments, submerged fermentation is used to grow engineered microbial cells using media containing soy carbon sources described herein. As used herein, the term “submerged fermentation” refers to a fermentation process in which the microorganisms can grow under the beneath the surface of the medium. Typically, liquid medium is used in submerged fermentation. In some embodiments, solid state fermentation is used to grow engineered microbial cells using media containing soy carbon sources described herein. As used herein, the term “solid state fermentation” refers to a fermentation process in which microorganisms can grow on the surface of the medium. Typically, solid medium is used in solid state fermentation. Examples of submerged fermentation and solid state fermentation are provided in the Examples section.

### Production of Industrial Chemicals

Engineered microorganisms as described herein can be used to produce any of a variety of products, in particular, those industrial chemicals. In certain embodiments, a microorganism provided herein produces a polypeptide, non-ribosomal peptide, acyl amino acid, and/or lipopeptide of interest (e.g., an acyl amino acid or lipopeptide which is a surfactant). As one non-limiting example, an engineered microorganism according to the present invention is used to produce surfactin.

In certain embodiments, an engineered microorganism is also engineered to produce a product of interest. For example, in some embodiments, a microorganism is engineered to express a polypeptide(s) that participates in the synthesis of the product of interest. In some embodiments, the polypeptide is an engineered polypeptide. In some embodiments, a microorganism that produces an acyl amino acid includes an engineered polypeptide comprising a fatty acid linkage domain, a peptide synthetase domain, and a thioesterase domain. In some embodiments, a microorganism that produces an acyl amino acid includes an engineered polypeptide comprising a fatty acid linkage domain, a peptide synthetase domain, and a reductase domain. In various embodiments, one or more of the fatty acid linkage domain, the peptide synthetase domain, and the thioesterase domain are surfactin synthetase domains. Methods of producing lipopeptides and acyl amino acids using engineered polypeptides, and methods of producing microorganisms that include the polypeptides are described in WO 2008/131002 and WO 2008/131014, the entire contents of which are hereby incorporated by reference.

In certain embodiments, a microorganism used to produce a polypeptide, non-ribosomal peptide, acyl amino acid, and/or a lipopeptide of interest is a bacterium. Non-limiting examples of bacteria that can be grown in accordance with the present disclosure include bacteria of the genera Bacillus, Clostridium, Enterobacter, Klebsiella, Micromonospora, Actinoplanes, Dactylosporangium, Streptomyces, Kitasatospora, Amycolatopsis, Saccharopolyspora, Saccharothrix and Actinosynnema. In certain embodiments, a microorganism used to produce a polypeptide, non-ribosomal peptide and/or a lipopeptide in accordance with the present disclosure is a bacterium of the genus Bacillus. In certain embodiments, a microorganism used to produce a polypeptide, non-ribosomal peptide, acyl amino acid, and/or a lipopeptide in accordance with the present disclosure is a bacterium of the species Bacillus subtilis. One skilled in the art will understand that other bacteria can be engineered according to the present invention to produce polypeptides, non-ribosomal peptides, acyl amino acids, and/or lipopeptides.

In certain embodiments, the yield of a polypeptide, non-ribosomal peptide, acyl amino acid, and/or a lipopeptide of interest produced by engineered microorganisms grown under conditions described herein is at least about 5%, 10%, 15%, 20%, 25%, 30%, 35%, 40%, 45%, 50%, 55%, 56%, 57%, 58%, 59%, 60%, 65%, 70%, 75%, 80%, 85%, 90% or more. Yield is defined as the amount of carbon source (e.g., soy molasses) that is converted to product (e.g., a polypeptide, non-ribosomal peptide, acyl amino acid, and/or a lipopeptide). Thus, if 50% of the carbohydrates present in soy molasses is converted to a polypeptide, non-ribosomal peptide, acyl amino acid, and/or a lipopeptide, the yield is 50%.

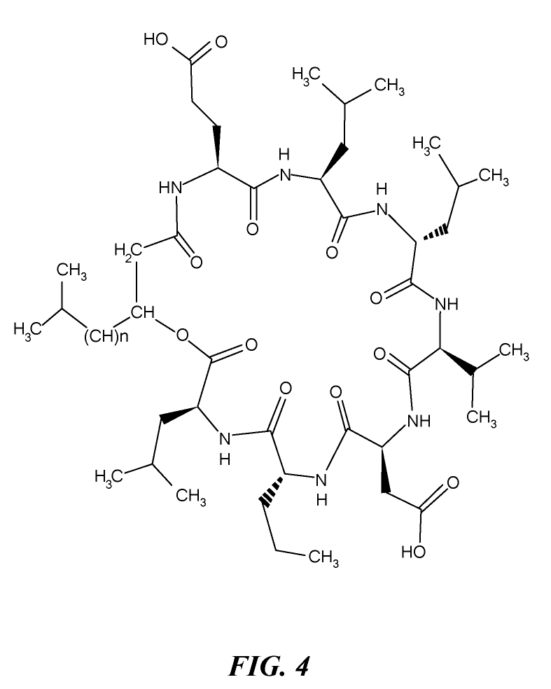

The present invention is particularly useful in producing surfactants. In some embodiments, the present invention provides engineered Bacillus subtilis that produces a biosurfactant called surfactin (FIG. 4). Surfactin is one of the most powerful biosurfactants. It has been shown to reduce the surface tension of water from 72 mN/m to 27 mN/m at a concentration of 20 μM (Peypoux F, Bonmatin J M, Wallach J., Appl. Microbiol. Biotechnol., 1999, 51(5):553-63). Although this is impressive and surfactin has been readily available for over thirty years (Arima K, Kakinuma A, Tamura G., Biochem Biophys Res Commun., 1968, 31(3):488-94), it has not yet been launched as a commercial product. Surfactin has limited utility for many commercial products applications because of its low water solubility. We have used an automated microbial strain engineering system to produce a Bacillus strain that secretes a surfactin-derivative that is highly water soluble. See, International Patent application PCT/US2008/060474, Publication number WO2008131002, 2008.

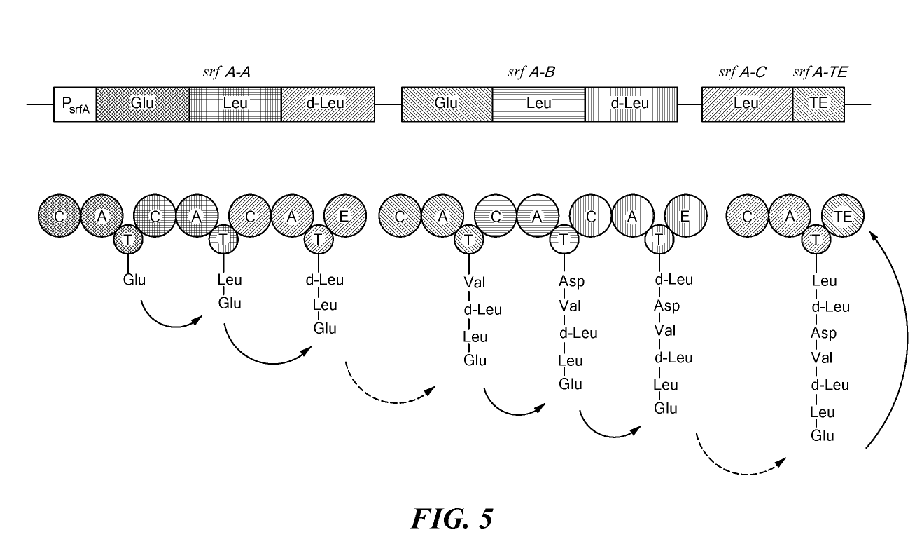

Surfactin is a cyclic lipopeptide synthesized by a peptide synthetase (FIG. 5), a multi-enzyme complex encoded by the srf operon (Stachelhaus T., Marahiel M A., FEMS Microbiology Letters, 1995, 125:3-14). The operon consists of many genes though three are of primary interest: srfA-A, srfA-B, and srfA-C. These three genes work together to assemble surfactin by stepwise assembly of amino acids. In the first step of the process, the lipid component becomes linked to the first amino acid (Glu) of surfactin. The other six amino acids of surfactin are added one-by-one to the growing polymer, and the final product is released via the action of the terminal thioesterase domain (TE) which catalyzes lactone bond formation between the terminal amino acid of the surfactin molecule and the β-hydroxyl of the fatty acid chain.

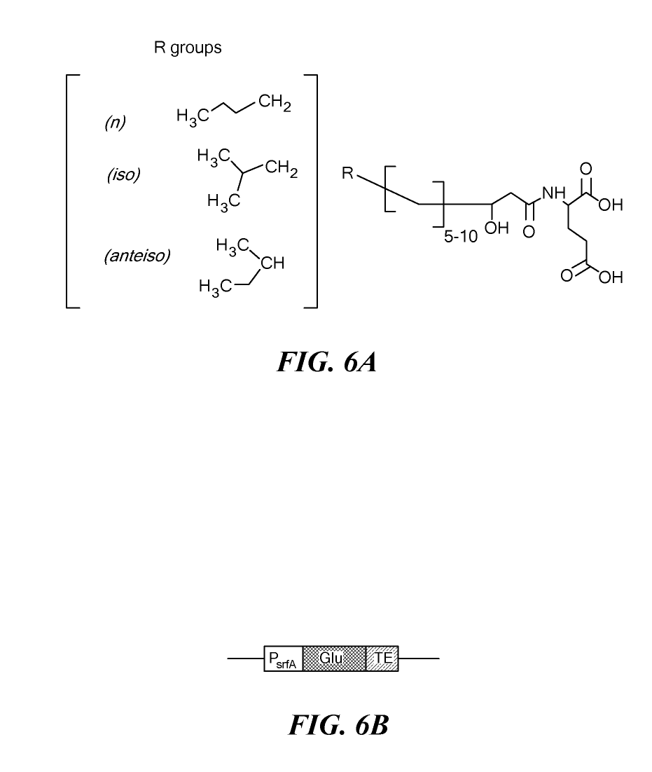

In order to produce a water soluble surfactant, we radically reduced the size of the synthetase by eliminating all hydrophobic amino acids, deleting about 27 kilobases (kb) of the Bacillus genome in order to make the gene variant shown in FIG. 6b, which produces β-hydroxy myristoyl glutamate, referred to hereafter as FA-Glu (fatty acid-glutamate). See, International Patent Application PCT/US2008/060474, Publication Number WO2008131002, 2008.

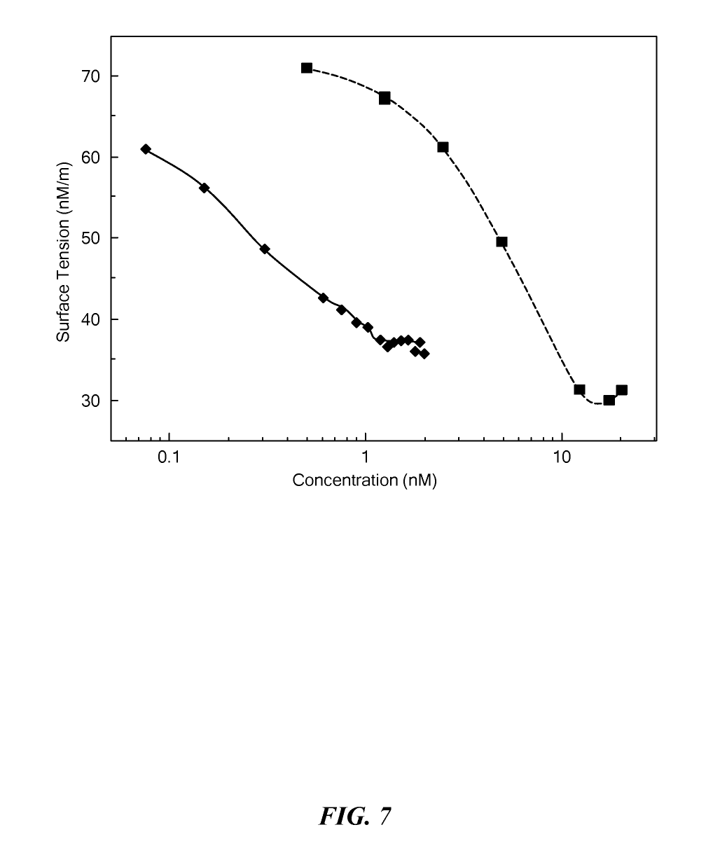

FA-Glu is similar to a commercial product that is already on the market, myristoyl glutamate, which is manufactured and sold by Ajinomoto and other companies. FA-Glu produced by strains described herein was found to have a lower critical micelle concentration than myristol glutamate (FIG. 7). Myristoyl glutamate is used in many personal care products (Husmann M., http://www.in-cosmetics.com/ExhibitorLibrary/420/2007-05b_PERLASTAN_Surfactants_{—}3.pdf), and can be used in over-the-counter drug formulations such as contact lens solutions (Castillo et al, U.S. Pat. No. 6,146,622). It is manufactured by a chemical process in which an amino acid (produced by fermentation) is linked to a fatty acid, which is derived from vegetable oil, such as palm oil or coconut oil. Although the commercial product itself is “green” it is manufactured using raw materials that are produced in a manner that threatens the rainforest and leads to increased carbon dioxide emission (United Nations Development Programme, Palgrave Macmillan, 2007. http://hdr.undp.org/en/media/HDR_{—}20072008_EN_Complete.pdf).

We examined whether FA-Glu producing strain would grow on media with soy molasses as the sole carbon source but would not be able to completely utilize it. It was found that the FA-Glu producing strain did grow on media with 0.5% soy molasses as the sole carbon source. The productivity of FA-Glu was 108.8 mg/L after 3 days.

In some embodiments, the present invention provides engineered strains (e.g., engineered Bacillus subtilis strains) in which the volumetric productivity of a surfactant (e.g., FA-Glu) is increased and allows the strains to efficiently utilize some or all carbohydrates in soy components (e.g., soy molasses, soy meal or soy hulls) such as raffinose, stachyose and verbascose. By cleaving the glycosidic bonds in the galacto-oligosaccharides enzymatically, simple sugars become available and can be utilized as a carbon source to support cell growth and surfactant production. The strains enable cost effective production of surfactants and other chemicals using soy components (e.g., soy molasses and/or soy hulls) as the feedstock.

There exist (in organisms other than Bacillus subtilis) α-galactosidase enzymes that specifically digest the oligosaccharides stachyose and raffinose to produce smaller sugars by cleaving the α-1-6 links between the sugar units (Rehms, H., Barz W., Appl Microbiol Biotechnol, 1995, 44: 47-52). Raffinose is broken down to sucrose and galactose by these enzymes. In some embodiments, the present invention provides methods for introducing one or more of these heterologous α-galactosidase enzymes into Bacillus, to produce an engineered strain able to cleave galacto-oligosaccharides to produce sucrose and galactose. Bacillus subtilis encodes an enzyme (sucrase (EC 3.2.1.26)) that cleaves the β-1-2 bonds in sucrose (Prestidge L S, Spizizen J., 1969, 59(2):285-8. http://mic.sgmjournals.org/cgi/reprint/59/2/285?view=long&pmid=4984180), generating fructose and galactose. Fructose can be taken-up and metabolized by Bacillus subtilis. In addition, if a galactose permease is introduced into the Bacillus strain, galactose can be take-up, and subsequently metabolized by enzymes from the galactose operon (genes galK, galT and galE) (Krispin O., Allmansberger R., J Bacteriol, 1998, 180(8):2265-2270. http://jb.asm.org/cgi/content/full/180/8/2265?view=long&pmid=9555917) to feed into the glycolytic pathway.

It is well documented that raw material costs can contribute as much as 75% of the cost of production of fermentation product (Lynd L R, Wyman C E, Gerngross T U., Biotechnol Prog., 1999, 15(5):777-793). Soy molasses sells for about ⅕^{th} the price of glucose. By using soy molasses as the feedstock, it is possible to achieve commercial profit with an engineered strain that has a lower productivity than what would be required using glucose as the feedstock. For example, by one estimate, volumetric productivity required to enable commercial-scale production of FA-Glu is about 0.6 grams per liter per day, if soy-molasses is used as the feedstock. Using strain engineering to increase the efficiency of utilization of soy molasses, the volumetric productivity can be increased even further. In some embodiments, strains and methods herein permit a volumetric productivity of at least 0.6 g/L/day, 0.8 g/L/day, 1.0 g/L/day, 1.2 g/L/day, or more. In some embodiments, using engineered strains and methods described herein, a fermentation process may convert at least 10%, 15%, 20%, 25%, 30%, 35%, 40%, 45%, 50%, 55%, 56%, 57%, 58%, 59% 60% or more of the soy carbon source into FA-Glu.

### EXAMPLES

### Example 1

### Engineering of B. subtilis to Overexpress Certain Glycosidases

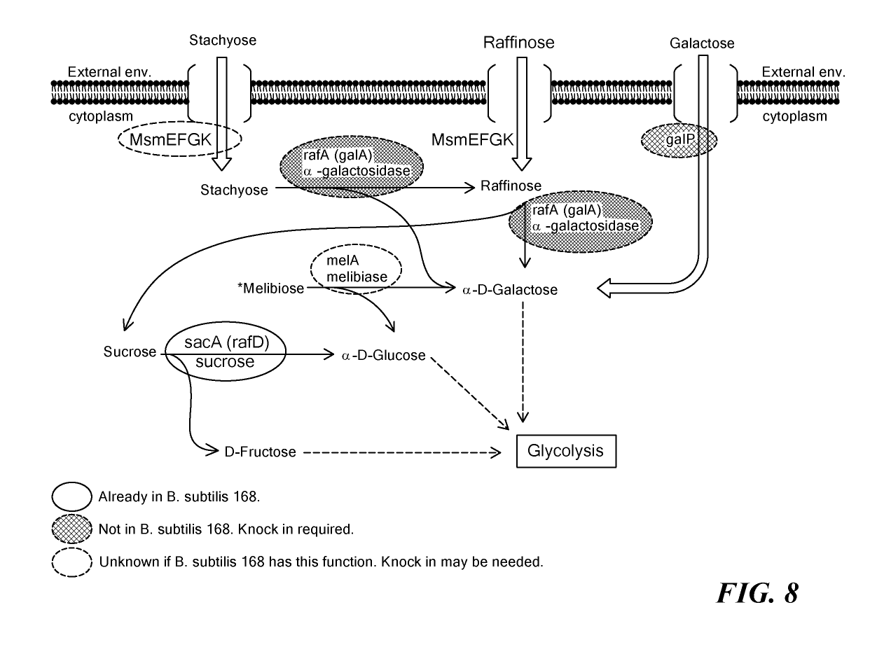

The present example describes one exemplary strategy for engineering microorganisms such as B. subtilis for more efficient utilization of soy carbohydrates (FIG. 8). The putative melibiase enzyme of Bacillus subtilis, encoded by the melA gene, putatively capable of hydrolyzing the α-1-6 links specifically in the disaccharide melibiose, can be used to cleave the galactose-glucose linkages in melibiose, stachyose and raffinose (Oh Y K et al, J. Biol. Chem., 2007, 282(39): 28791-28799. http://www.jbc.org/cgi/content/full/282/39/28791). To examine its activity, this putative melibiase enzyme is overexpressed, purified and assayed on the three sugars in vitro.

B. subtilis is engineered to overexpress melibiase using methods we have previously described. (See, e.g., Fabret et al. (2002) “A new mutation delivery system for genome-scale approaches in Bacillus subtilis”, Mol. Microbiol., 46:25-36 and International Patent Application Serial No. PCT/US2008/060474 (published as WO2008131002), the entire contents of each of which are incorporated herein by reference.) Specifically, a construct encoding melA is generated by standard genetic engineering techniques. This construct is then integrated into the chromosome of B. subtilis by homologous recombination as described previously.

Alternatively or additionally, a variant of α-galactosidase that has a strong demonstrated ability to cleave raffinose and stachyose can be expressed in Bacillus subtilis cells using similar methods described herein. Alternatively or additionally, the rafA gene of Escherichia coli can be used (Aslanidis C., Schmid K., Schmitt R., J. Bacteriology, 1989, 6753-6763. http://jb.asm.org/cgi/reprint/171/12/6753?view=long&pmid=2556373).

### Example 2

### Conditions for Submerged Fermentation of B. subtilis

In the present Example, strains of B. subtilis were grown and fermented using soy carbon sources according to a liquid growth (“submerged”) protocol for production of FA-Glu and of surfactin.

Cultures were grown in liquid volumes ranging from 10 mL in 50 mL conical tubes, 50 mL in 250 mL E-flasks, 500 mL in 2 L E-flasks and 8 L in 12 L benchtop fermenters. Liquid media used were variants of either MM15 or S7 (recipes below) with a variety of carbon sources including soy products and cellulosic intermediates. Strains of interest were generally grown to saturation in M9YE media and then seeded at 2% into medium formulation. Fermentation cultures were grown at either 30° C. or 37° C. with agitation for 3-5 days. Liquid samples were removed, insoluble materials were removed, and material were analyzed and quantified via LCMS.

### Media Compositions

| M9YE | 6 | g | Na₂HPO₄ |
|---|---|---|---|
| | 3 | g | KH₂PO₄ |
| | 0.5 | g | NaCl |
| | 1 | g | NH₄Cl |
| | 3 | g | Yeast Extract |
| | 0.5% | | Glucose |
| MM15 | 2 | g | Ammonium Sulfate |
| | 14 | g | K₂HPO₄ |
| | 6 | g | KH₂PO₄ |
| | 1 | g | Na₃ Citrate |
| | 0.2 | g | MgSO₄*7H₂O |
| | 4% | | Glucose |
| | 100 | μM | Calcium Chloride |
| | 4 | μM | Ferric Sulfate |
| | 4 | μM | EDTA |
| | 10 | μM | MnSO₄ |
| S7 | 100 | mM | Potassium Phosphate pH 7.5 |
| (Phos7.5) | 10 | mM | Ammonium Sulfate |
| | 20 | mM | Glutamic Acid |

| 2% | | Glucose |
|---|---|---|
| 1:100 dilution of Trace Metals | | |
| Solution (1 L recipe below) | | |
| 2 | mL | 1M HCl |
| 40.6 | g | MgCl₂*6H₂O |
| 1.47 | g | CaCl₂*2H₂O |
| 0.99 | g | MnCl₂*4H₂O |
| 13.6 | mg | ZnCl₂ |
| 135 | mg | FeCl₃*6H₂O |
| 67.5 | mg | Thiamine-HCl |

### Soy Carbon Sources

Soy molasses was obtained from Archer Daniels Midland Co (ADM) and assumed to be 10% solids. In some experiments, soy molasses was used at a concentration of 0.5% soy molasses (1:20 dilution of raw material).

Soy meal was obtained from Zeeland Soya (47% Protein, 1% Fat, 3.5% Fiber). Soy meal was autoclaved in RO-di-H₂O before use in fermentations. In some cultures, solid materials remained throughout fermentation. In some cultures, a soy meal extract was used as the soy source. To make this extract, soy meal was autoclaved at a higher concentration (i.e., 8%) and liquid soluble portion was removed, re-autoclaved and diluted to desired concentration in liquid media (i.e., 0.5%). In some experiments, soy meal extract was used at a concentration of 0.5%; cultures were grown at 37° C. for 3 days after addition of soy meal extract.

Crushed soy hull was obtained from US Soy (18-23% protein, 5-10% fat, 55-65% carbohydrate, 50-65% fiber). Simultaneous saccharification and fermentation was set up using combinations of enzyme treatment with cellulase, beta-glucosidase, hemicellulase and/or pectinase. Hulls were used at either 2% or 8% solids.

### Example 3

### Conditions for Solid State Fermentation of B. subtilis

In the present Example, engineered strains of B. subtilis were grown and fermented using soy carbon sources according to a variety of solid state fermentation (SSF) protocols in order to optimize conditions for fermentation and production of products of interest. Solid state fermentation may allow reductions in the time and resources used to ferment large quantities of B. subtilis.

Ground soybean hulls were autoclaved either dry or with varying amounts of water, and varying concentrations of S7 components and cells were added after autoclaving. In some conditions cell growth was observed on the surface of this “tray” fermentation after about 48-72 hours growth without agitation. Growth is observed by a whitish cell color and a purple haze that tends to accompany surfactant production. Cell growth is typically observed at the solid-air interface, therefore a very uniform thin layer of soy hull and cells may be desirable.

Experiment 1

10 g ground soy hull (Minnesota Soybean Processors) was autoclaved in either 25, 50 or 100 mL (2.5:1, 5:1, 10:1) water (15 min 121° C.) inside Matrix 1250 μL pipet tip boxes. S7 (phosphate 7.5, no glutamic acid, no glucose) components were added to a 1× final concentration. Seed culture (23960-A1) was grown overnight to saturation in M9YE (no Glucose) at 37° C. and used at 2%. Cultures grown at 37° C. with no shaking for 68.5 hr. A tray of water was used in the incubator for increased humidity and/or less evaporation.

Soy hull and liquid portions were scooped into 200 mL Nunc conical tubes and centrifuged at 5000×g for 10 minutes to remove residual water. Soy hulls were washed with 20 mL ˜99.9% methanol (with the pH adjusted to 9.6 with 1M NaOH) and incubated for ˜15 minutes at room temperature. Soy hulls and methanol were centrifuged at 5000×g for 5 minutes, and the methanol removed. Hulls were washed a second time as before, and the methanol removed. Hulls were washed a third time with 30 mL methanol, incubated for ˜45 minutes, and centrifuged; methanol was removed.

For quantification, 0.5 mL samples of liquid fractions were dried in a speedvac and resuspended in 0.5 mL water. Samples were centrifuged at 13,000 rpm for 5 minutes to remove any insoluble material. 0.4 mL of supernatant was filtered through 0.45 μm spin columns at 7000×g for 1 minute. Samples were diluted either 1:50 or 1:100 for LCMS analysis and quantified using internal standards.

Experiment 2 (Variation of Experiment 1)

5 g ground soy hull was autoclaved with or without 25 mL (5:1) water, with or without 0.3% agarose, with or without 2% soy meal. (In some cases water was added after autoclaving.) S7 components were added to a final concentration 1×. Seed culture was grown overnight to saturation in M9YE (0.5% glucose) at 37° C. and used at 2%. Cultures were grown as above for approximately 95 hrs.

Soy hull and liquid portions were collected as above. Soy hulls were washed with either 20 mL ˜100% Ethanol or water (pH adjusted to 9.9 with 1M NaOH) and incubated for ˜15 minutes at room temperature. Hulls and ethanol/water were centrifuged at 5000×g for 5 minutes; liquids were removed. Hulls were washed three additional times and liquids were removed after each wash.

For quantification, 0.5 mL samples of liquid fractions were processed and analyzed as described for Experiment 1.

Experiment 3 (Protocol with Shaking in Conical Tubes)

2.5 g ground soy hull was autoclaved with or without 20 mL (8:1) water, with or without 2% soy meal, with or without 1% soy molasses (1:10 dilution of ADM stock material) in 50 mL conical tubes. (In some cases water added after autoclaving.) S7 components were added to a final concentration of 1× or a 1:10 or 1:100 dilution. Seed culture was grown as described above. Cultures were grown at 37° C. with shaking for approximately 94 hr.

Soy hulls were washed with 15 mL water (pH adjusted to 9.5 with 1M NaOH) and incubated for ˜15 minutes at room temperature. Hulls and water were centrifuged at 5000×g for 10 minutes; liquids were removed. Hulls were washed two additional times with water as described above and once with 100% ethanol; liquids were removed after each wash.

For quantification, 0.5 mL samples of liquid fractions were processed and analyzed as described for Experiment 1.

Experiment 4 (Variation of Experiment 2)

5 g ground soy hull was autoclaved with 25 mL (5:1) water, with or without 2% soy meal, with or without 1% soy molasses. S7 components were added to a final concentration of 1×. Seed culture was grown as described above. Cultures grown as described above for approximately 73 hr.

Soy hull and liquid portions were collected as above. Soy hulls were washed with 20 mL water (pH adjusted to 9.5 with 1M NaOH) and incubated for ˜15 minutes at room temperature. Hulls and water were centrifuged at 5000×g for 5 minutes; liquids were removed. Hulls were washed two additional times with water as described above and once with 100% ethanol; liquids were removed after each wash.

For quantification, 0.5 mL samples of liquid fractions were processed and analyzed as described for Experiment 1.

Experiment 5 (Variation of Experiment 4 with Supplementation)

5 g ground soy hull was autoclaved with 25 mL (5:1) water, 2% soy meal, and 1% soy molasses. S7 components were added to a final concentration of 1×. Seed culture was grown as described above. Cultures were grown at either 37° C. or 42° C. for ˜45 hr and then supplemented with either 2% soy meal extract, 1% soy molasses, or a combination thereof. Cultures were grown an additional ˜73 hr after supplementation.

Soy hull and liquid portions were collected as above. Soy hulls were washed with 20 mL water (pH adjusted to 9.5 with 1M NaOH) and incubated for ˜15 minutes at room temperature. Hulls and water were centrifuged at 5000×g for 5 minutes; liquids were removed. Hulls were washed three additional times with water as described above; liquids were removed after each wash.

For quantification, 0.5 mL samples of liquid fractions were processed and analyzed as described for Experiment 1.

Experiment 6 (Variation of Experiment 5 with Mixing)

5 g Ground Soy Hull was autoclaved with 25 mL (5:1) water, 2% soy meal, and 1% soy molasses. S7 components were added to a final concentration of 1×. Seed culture was grown as described above and added at 2% or as a 10× cell concentrate. Cultures grown at 37° C. for ˜44 hr and then supplemented with either 2% soy meal extract, 1% soy molasses, or a combination thereof. Cultures were grown an additional 24 hr after supplementation. Cultures were mixed daily with a pipet tip.

Soy hull and liquid portions were collected as above. Soy hulls were washed with 20 mL water (pH adjusted to 9.5 with 1M NaOH) and incubated for ˜15 minutes at room temperature. Hulls and water were centrifuged at 5000×g for 5 minutes; liquids were removed. Hulls were washed three additional times with water as described above; liquids were removed after each wash.

For quantification, 0.5 mL samples of liquid fractions were centrifuged at 13000 rpm for 5 minutes to remove insoluble material. 0.4 mL of supernatant was filtered through 0.45 μm spin columns at 7000×g for 1 minute. Samples were diluted either 1:50 or 1:100 for LCMS analysis and quantified using internal standards.

### Example 4

### Production of FA-Glu Using Engineered B. subtilis Strains

In the present Example, strains of B. subtilis are engineered to express a glucosidase that may increase the efficiency of the strains in utilizing soy carbon sources.

Several strains of B. subtilis are used as “starting strains”: strains 28836, 23960-A1, and 34170-E1. These strains harbor modifications that improve general robustness of the strains and/or that allow or enhance production of either surfactin or FA-Glu. Strain 28836 is a modified version of OKB 105, which is a variant of BS168 that has had its sfp gene restored such that it produces surfactin. Strain 28836 has a restored phenylalanine gene to make the cells more robust. Strain 23960-A1 is a modified version of OKB105 in which modules 2-7 of the surfactin synthetase is removed, such that the cells produce FA-Glu. See, e.g., International Patent Application Serial No. PCT/US2008/060474 (published as WO/2008/131002); International Patent Application Serial No. PCT/US09/58061 (published as WO/2010/036717) and International Patent Application Serial No. PCT/US09/58049 (published as WO/2010/039539), the entire contents of each of which are incorporated herein by reference.

In the present Example, these strains are further modified in order to increase utilization of soy carbon sources.

A rafA gene from Escherichia Coli is expressed in these strains. rafA encodes an α-galactosidase and is part of an operon that encodes functions required for inducible uptake of raffinose. rafA is stably introduced into each of the starting strains by standard techniques as previously described. Strains stably expressing rafA are isolated and stored and/or cultured for analyses and further manipulations.

Engineered strains are grown and fermented according to conditions similar to those described in Examples 2 and/or 3 to produce FA-Glu. Yields of surfactin or FA-Glu are analyzed to assess productivity of engineered strains.

### Example 5

### Assays for Production of FA-Glu and for Utilization of galacto-oligosaccharides

This example describes assays that can be used to monitor FA-Glu production and to determining the efficiency of utilization of galacto-oligosaccharides. Specifically, FA-Glu has been monitored by reversed phase high performance liquid chromatography (RP-HPLC) on a C18 Hypersil Gold column (50×2.1 mm, particle size 1.9 μm) with mass spectrometry (MS) detection using a Thermo-Scientific Accela high-speed LC system coupled to a Thermo Scientific LXQ ion trap mass spectrometer. Chromatographic separation was achieved by gradient elution with acetonitrile and water containing 1% of acetic acid, and the FA-Glu molecules were detected at m/z 344.21, 358.22, 372.24, 386.25, 400.27 and 414.28 in the negative ion mode. For the quantitative analysis, the purified FA-Glu was used as the standard to construct a calibration curve. The amount of FA-Glu in the unknown sample was measured using the derived correlation equation between the LC-MS peak area and sample concentration.

Utilization of the carbohydrates in soy molasses can be determined by high performance anion exchange chromatography-pulsed amperometric detection (HPAEC-PAD) (Qureshi N, Lolas A, Blaschek H P, J Ind Microbiol Biotechnol., 2001, 26(5):290-5). The separation will be performed on a CarboPac PA1 analytical column using 0.1M NaOH as mobile phase. The sugars elute based on their size, composition and linkage and are then detected by a pulsed electrochemical detector. Standards of glucose, galactose, fructose, sucrose, melibiose, raffinose, and stachyose will be used for the identification and quantification of the residual sugars.

Alternatively, the quantity of the major soy oligosaccharides-stachyose and raffinose as well as their degradation products can also be measured by LC-MS using a Hypercarb porous graphitized carbon column. Due to the polar retention effect of porous graphitized carbon, this column provides excellent chromatographic separation for difficult-to-retain polar compounds such as oligosaccharides (Robinson S, et al., Anal. Chem., 2007, 79:2437-2445). Hypercarb can also be used for RP-HPLC with acetonitrile-water as the eluent, which is suitable for MS detection.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. The articles “a”, “an”, and “the” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses variations, combinations, and permutations in which one or more limitations, elements, clauses, descriptive terms, etc., from one or more of the claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth herein. It should also be understood that any embodiment of the invention, e.g., any embodiment found within the prior art, can be explicitly excluded from the claims, regardless of whether the specific exclusion is recited in the specification.

It should also be understood that, unless clearly indicated to the contrary, in any methods claimed herein that include more than one act, the order of the acts of the method is not necessarily limited to the order in which the acts of the method are recited, but the invention includes embodiments in which the order is so limited. Furthermore, where the claims recite a composition, the invention encompasses methods of using the composition and methods of making the composition. Where the claims recite a composition, it should be understood that the invention encompasses methods of using the composition and methods of making the composition.

### INCORPORATION OF REFERENCES

All publications and patent documents cited in this application are incorporated by reference in their entirety to the same extent as if the contents of each individual publication or patent document were incorporated herein.

What is claimed is:

1. An engineered microbial cell comprising a modification that increases efficiency of utilization of a soy carbon source as compared with a parent cell.

2. The engineered cell of claim 1, wherein the soy carbon source is soy molasses, soy meal, soy hulls and/or an extract thereof.

3. The engineered cell of claim 2, wherein the soy carbon source is a cellulosic component present in the soy molasses, soy meal, soy hulls and/or the extract thereof.

4. (canceled)

5. The engineered cell of claim 1, wherein the modification comprises altered expression or activity of a carbohydrate modifying enzyme.

6. The engineered cell of claim 5, wherein the altered expression or activity is increased expression or activity.

7. (canceled)

8. The engineered cell of claim 1, wherein the modification comprises altered localization of a carbohydrate modifying enzyme.

9-11. (canceled)

12. The engineered cell of claim 5, wherein the carbohydrate modifying enzyme is selected from the group consisting of melibiases, α-galactosidases, β-fructosidases, exoglucanases, acetyl esterases, α-glucuronidases, endoglucanases, cellobiohydrolases, xylanases, beta-xylosidases, alpha-L-arabinofuranosidases, acetyl xylan esterases, mannanases, xyloglucanases, polygalacturonases, exo-beta-1,3-glucosidases, lignin peroxidases, and combination thereof.

13-18. (canceled)

19. The engineered cell of claim 1, wherein the modification comprises increased expression or activity of a saccharide transporter.

20. (canceled)

21. The engineered cell of claim 1, wherein the cell is a bacterial cell.

22. The engineered cell of claim 21, wherein the bacterial cell is selected from the group consisting of Bacillus, Clostridium, Enterobacter, Klebsiella, Micromonospora, Actinoplanes, Dactylosporangium, Streptomyces, Kitasatospora, Amycolatopsis, Saccharopolyspora, Saccharothrix, Actinosynnema and combination thereof.

23. The engineered cell of claim 22, wherein the bacterial cell is a Bacillus cell.

24. The engineered cell of claim 23, wherein the Bacillus cell is a Bacillus subtilis cell.

25. The engineered cell of claim 1, wherein the cell is further engineered to produce a product of interest.

26. The engineered cell of claim 25, wherein the product of interest is selected from the group consisting of a polypeptide, a non-ribosomal peptide, an acyl amino acid, a lipopeptide and combination thereof.

27. (canceled)

28. The engineered cell of claim 26, wherein the lipopeptide is a surfactin.

29. The engineered cell of claim 26, wherein the lipopeptide is FA-Glu.

30. A fermentation process comprising growing an engineered microbial cell of claim 1 in a culture medium comprising a soy carbon source.

31. The fermentation process of claim 30, wherein the soy carbon source comprises soy molasses, soy meal, soy hulls, an/or an extract thereof.

32. The method of claim 30, wherein the medium lacks a carbon source other than the soy carbon source.

33. The fermentation process of claim 30, wherein the fermentation process is a submerged fermentation process.

34. The fermentation process of claim 30, wherein the fermentation process is a solid state fermentation process.

35. The fermentation process of claim 30, wherein the fermentation process converts at least 10% of the soy carbon source into chemical products.

36. A method of producing an industrial chemical comprising
growing an engineered microbial cell in a culture medium comprising a soy carbon source,
wherein the engineered microbial cell comprises a modification that increases efficiency of utilization of the soy carbon source as compared with a parent cell, and further wherein the engineered microbial cell produces an industrial chemical of interest.

37. The method of claim 36, wherein the soy carbon source comprises soy molasses, soy meal, soy hulls, an/or an extract thereof.

38. The method of claim 36, wherein the culture medium lacks a carbon source other than the soy carbon source.

39. The method of any one of claims 36, wherein the engineered microbial cell is an engineered Bacillus subtilis cell.

40. The method of claim 36, wherein the industrial chemical of interest is selected from the group consisting of a polypeptide, a non-ribosomal peptide, an acyl amino acid, a lipopeptide and combination thereof.

41. The method of claim 40, wherein the industrial chemical of interest comprises a lipopeptide.

42. The method of claim 41, wherein the lipopeptide is a surfactin.

43. The method of claim 42, wherein the lipopeptide is FA-Glu.
